(19) United States
(12) Patent Application Publication
(10) Pub. No.: US 2018/0346802 A1
(43) Pub. Date: Dec. 6, 2018

(54) Hydraulic Fracturing Fluid

(71) Applicant: Noles Intellectual Properties, LLC, Washington, OK (US)

(72) Inventors: Jerry W. Noles, JR., Blanchard, OK (US); Alex Jason Watts, Rayville, LA (US)

(73) Assignee: Noles Intellectual Properties, LLC, Washington, OK (US)

(21) Appl. No.: 15/614,244

(22) Filed: Jun. 5, 2017

(51) Int. Cl. C09K 8/88; C07C 309/03

(52) U.S. Cl. CPC C09K 8/882; C08L 29/04; C07C 309/03; C09K 8/887

(57) ABSTRACT

A polymer and hydraulic fracturing fluid provide an improved ability to transport proppant. In an embodiment, a method of designing a hydraulic fracturing fluid includes calculating an elastic modulus of a polymer. The method also includes calculating a critical velocity of a hydraulic fracturing fluid comprising the polymer and proppant based on the elastic modulus of the polymer.

### BACKGROUND

Within recent years, the oil and gas industry has developed the use of hydraulic fracturing to produce what was once considered nonproductive oil and gas formations. Typically, most of these formations are made up of low or no porosity shale wherein the oil and gas may exist within micro-fractures present in these formations and not within the shale itself. For this reason, connecting more of these micro fractures and keeping them connected to each other and to the wellbore may be beneficial to the successful and continuous production of the well. The degree of fluid communication though the fractured formation and wellbore may be referred to as conductivity of the formation. This hydraulic fracturing technology may include the use of high volumes of water and a propping agent to be pumped into subterranean wells under tremendous rates and pressures to pry rock apart, thereby allowing the oil and gas that is trapped within the matrix of the oil and gas formations to migrate to the wellbore and production casing. Although the use of this technology may have allowed high volumes of oil and gas recovery, there exists challenges with transport of propping agents through long wellbore conduits.

Hydraulic fracturing is typically based on three fundamental elements. First, drill horizontally within the reservoir to expose long sections of the oil and gas bearing formation to the pressure conduit. Two, secure the pressure conduit in place using cement or packers, then perforate and isolate 100 to 250 foot sections of the conduit generating a passage from the conduit to the reservoir. Third, using water, sand and pressure, pump through the perforated channels prying the rock apart, then use the sand to prop the formation open and allow the oil and gas to migrate into the well. Thereafter, repeat the process until the entire length has been connected to the reservoir. Although fracturing may be basic in concept, optimizing the process has proven challenging and is continuously evolving.

There are several issues related to the hydraulic fracturing process that may become problematic when wellbores reach extreme lengths. One challenge is that the pressure that the water needs to be pumped at has to be sufficiently high enough to continuously exert a force on the rock sufficient to pry the rock apart or to exceed the fracture gradient of the reservoir. This force is often needed to be continuously maintained as the water penetrates the reservoir and radiates away from the wellbore. Therefore, the flow rate of fracturing fluid has to be maintained high enough to continuously exert sufficient force to progress the fracture even as the area increases and as leak off occurs as the leading edge of the fracture encounters micro fractures in the formation. Since the oil and gas is believed to exist within these micro-fractures, the more micro-fractures that may be connected to the flow of water and sand the more productive the well may potentially be. However, once the rate of leak off of fluid into the micro-fractures becomes equal to the rate of fluid that is being pumped from surface, the ability to apply enough force to separate the rock and progress the fracture radially from the main borehole may become lost.

Reducing the friction during pumping may decrease the pressure drop due to friction thereby delivering greater force to the reservoir resulting in higher radial coverage and increased micro-fracture connectivity. Chemicals such as polyacrylamides have been used in reducing pump friction by lowering the interfacial surface tension between the fluid and the conduit. This lower interfacial surface tension may allow for lower pump pressures and higher rates to be used. The process may become counterproductive when it comes to the transportation of the sand into the fractured cavity. If the fluid rates become too low, then the sand may be left far behind the leading edge of the fracture. The pump rates may be adequate to transport the sand though the conduit area, but once the sand exits into the formation and the area increases, the sand may stop progression into the fracture once the critical velocity drops below about 1,700 feet per minute.

Some fracturing techniques have incorporated the use of smaller sand particles such as 100 mesh sand to aid in particle transport deeper into the fracture cavity as the 100 mesh, sand may have a lower critical velocity. Other techniques to increase proppant transport includes using man-made proppants that have a lower specific gravity rendering it almost naturally buoyant, which may allow it to be floated into the well without the use of chemicals. However, the cost of these man-made proppants may often far exceed their benefit and use. Furthermore, smaller proppants have often proven problematic when it comes to plugging with contaminates from the water that can flow back from the well or even the chemicals themselves that are used to transport the sand.

In response to these challenges, rate and pressure in exchange have typically been sacrificed for sand placement near the leading edge of the fracture. Such sacrifice has historically been accomplished through the use of highly viscous crosslinked polymers such as guar crosslinked with a borate or metallic cation, which may create a 3-dimensional polymer structure with much higher suspension characteristics. These viscous mixtures are typically pumped alongside an oxidizing breaker such as persulphate to allow the viscous mixture to be thinned once it has transported the sand into position. However, as these polymer chains are broken, they may generate a solid mass or a precipitant that may damage the formation and may result in reduced permeability by plugging the pore space generated by the fracturing sand and hindering the well's production. The industry's use of viscous fluids to maintain the sand in a state of suspension for extended periods has often proven to be problematic and has sacrificed rate and caused pump horsepower requirements to be very high. These viscous fluids have also historically not functioned well in fluid systems that were not fresh water with a neutral pH, and crosslinked systems may be sensitive to metallic compounds present in the water.

Therefore, there exists a need in the art for a polymer that does not rely on viscosity and particle suspension ability to transport proppant during a fracturing operation.

### SUMMARY OF EMBODIMENTS

These and other needs in the art are addressed in an embodiment of a method of designing a hydraulic fracturing fluid. The method includes calculating an elastic modulus of a polymer. The method also includes calculating a critical velocity of a hydraulic fracturing fluid comprising the polymer and proppant based on the elastic modulus of the polymer.

These and other needs in the art are addressed in other embodiments by a method of hydraulic fracturing. The method includes calculating an elastic modulus of a polymer. The method also includes calculating a critical velocity of a hydraulic fracturing fluid comprising the polymer and a proppant based on an elastic modulus of the polymer. In addition, the method includes preparing the hydraulic fracturing fluid comprising the polymer and the proppant. Moreover, the method includes injecting the hydraulic fracturing fluid through a tubular and into a subterranean formation at a volumetric rate such that a fluid velocity in the tubular is at or above a critical velocity.

In addition, these and other needs in the art are addressed by an embodiment of a hydraulic fracturing fluid. The hydraulic fracturing fluid includes an acrylamide-based polymer having an elastic modulus greater than about 30 dyn/cm². The hydraulic fracturing fluid also includes water and a proppant.

### BRIEF DESCRIPTION OF THE DRAWINGS

These drawings illustrate certain aspects of some of the embodiments of the present disclosure and should not be used to limit or define the disclosure.

FIG. 1 illustrates a viscous and elastic response to an applied strain in a fluid.

FIG. 2 illustrates an elastic versus viscous modulus for a fluid.

FIG. 3 illustrates a bar graph of polymer concentration and its effect of fluid properties.

FIG. 4 illustrates the operation temperature range of a polymer.

FIG. 5 illustrates a Lissajou plot for a fluid comprising a polymer.

FIG. 6 illustrates an elastic modulus test for various polymers.

FIG. 7 illustrates a viscous modulus test for various polymers.

FIG. 8 illustrates a comparison between elasticities for the polymers of FIG. 6 and

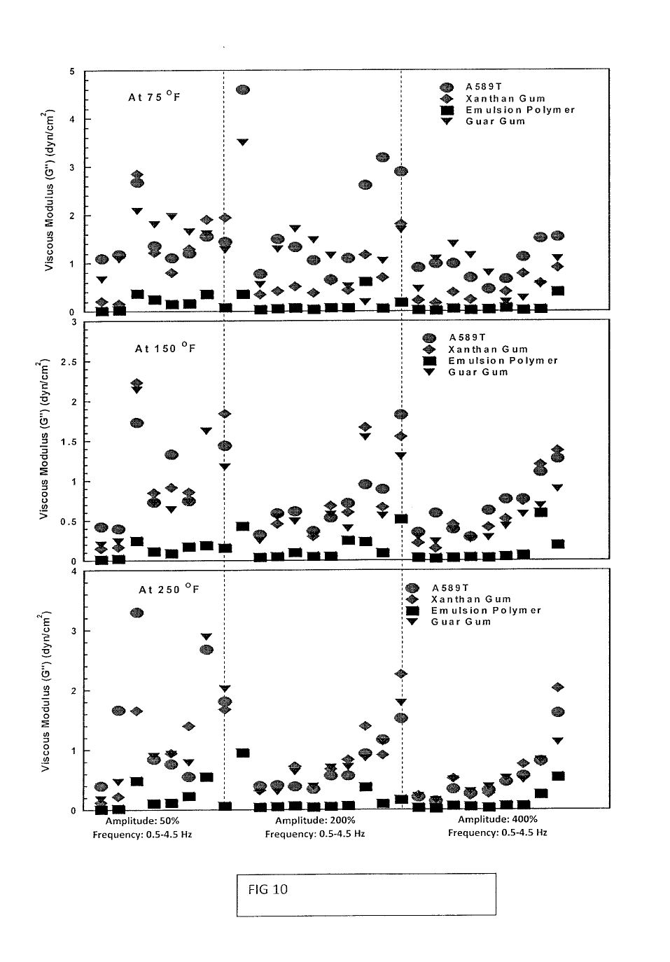

FIG. 7.

FIG. 9 illustrates an elastic modulus test for various polymers.

FIG. 10 illustrates a viscous modulus test for various polymers.

FIG. 11 illustrates a comparison between elasticities for the polymers of FIG. 9 and FIG. 10.

### DETAILED DESCRIPTION

The present disclosure may generally relate to a polymer that when hydrated has high viscoelastic properties. Furthermore, methods of designing a fracturing fluid comprising the polymer are provided. The present disclosure may also generally relate to hydraulic fracturing fluids wherein the fluid may comprise water and a soluble polymer that modifies rheological properties of the solution in which it is disposed.

In an embodiment, the polymer may have any suitable particle size. In embodiments, the polymer may comprise an average particle size determined by API sieving techniques of about 60 to about 100 US mesh size. Particle size may be an important factor to the rate of hydration of the polymer. In general, a smaller particulate size may hydrate quicker than a larger particulate size. Rapid hydration may be important so that the polymer may quickly act to suspend particulates and reduce friction, which may be especially important when pumping at relatively high rates such as 100 or more barrels per minute. Without rapid hydration, partial benefits of the polymer may be lost, and thus the polymer may be provided in a larger quantity to compensate for performance inefficiencies.

In an embodiment, the polymer may be provided as a dry granular material. The polymer may be directly introduced as a dry material into an aggressive moving body of fluid without high volume resonance requirements. Without limitation, the use of a dry material may reduce the cost as opposed to providing it as an oil based suspension, which may be typical oil based polymers presently in use. Further, without limitation, the requirements for transport and storage of a dry material are often less than providing as a liquid. Furthermore, the amount of polymer per unit volume of a dry polymer may be greater than what may be achieved with a liquid suspension (i.e. the moles of a polymer present in a volume of solid dry granules may be greater than the moles of a polymer present in the same volume of a liquid suspension).

In an embodiment, the polymer may comprise multiple repeating base units or monomers. In embodiments, the polymer comprises acrylamide, sodium amps (2-Acrylamido-2-methylpropane sulfonic acid), acrylic acid, sodium acrylate, vinyl alcohol, vinyl acetate, N-vinylpyrolidone, 2-hydroxymethy methacrylate, or any combinations thereof. In embodiments, the majority composition of the polymer may comprise acrylamide. In embodiments, acrylamide may be present in an amount of about 96.0 wt. % to about 99.0 wt. %. The polymer may be crosslinked with a cross linking agent. Any suitable cross linking agent may be used. In embodiments, cross linking agents include N,N′-methylenebis(acrylamide), boric acid, ethylene glycol diacrylate, polyethylene glycol diacrylate, or any combinations thereof. In embodiments, the cross linking agents may be present in an amount of about 0.1 wt. % to about 0.8 wt. %. Where used, the cross linker may also be combined with a reaction initiator. Any suitable reaction initiator may be used. In an embodiment, the reaction initiator comprises ammonium persulfate, benzoyl peroxide, potassium persulfate, sodium hypochlorite, polyethylene glycol diacrylate, or any combinations thereof. In embodiments, the reaction initiator may be present in an amount of about 0.005 wt. % to about 0.2 wt. %. Additionally, breakers may be used to cleave the cross linked bonds downhole. Any suitable breaker may be used. Suitable breakers may include ammonium persulfate, sodium persulfate, sodium hypochlorite, perborates, peroxides, enzymes, or any combinations thereof. In embodiments, a breaker may be present in an amount of about 200 ppm to about 600 ppm.

In some instances, the polymer may be damaged by the conditions of the wellbore. A protective agent may be used in conjunction with the polymer to protect the polymer from adverse temperature, pressure, and chemical species encountered in the wellbore or formation. Any suitable protective agents may be used. In embodiments, suitable protective agents include acrylamide, sodium amps (2-Acrylamido-2-methylpropane sulfonic acid), acrylic acid, sodium acrylate, vinyl alcohol, vinyl acetate, N-vinylpyrolidone, 2-hydroxymethy methacrylate, copolymerizations thereof, or any combinations thereof. A suitable protective agent may comprise AMPS (2-acrylamido-2-methylpropane sulfonic acid). With the addition of AMPS, it may be possible to use the polymer in more adverse pH conditions such as in a range of from about 1 to about 12 and with temperatures up to about 450° F. In embodiments, the protective agent may be present in an amount of about 5.0 wt. % to about 49.0 wt. %. Additionally, AMPS in conjunction with the polymer may be used in applications where total dissolved solids range from about 0 corresponding to fresh water or to saturation, for example a saturated brine.

In an embodiment, the dry granular material is added to provide a fracturing fluid with a polymer composition of between about 1.0 wt. % and about 50 wt. % polymer, alternatively between about 30 wt. % and about 40 wt. %, and alternatively about 35 wt. %.

Due to the wide operating range and conditions of the polymer and AMPS, the water used during fracturing may be from sources that are not freshwater such as produced water or well flow back fluids. The polymer and AMPS may be able to withstand being used in produced water, thereby reducing the freshwater requirement of the fracturing fluid.

Without being limited by theory, viscoelastic material may comprise both viscous and elastic properties. It is to be understood that in a perfectly elastic material, the stress and strain occur in phase so that the response of one occurs simultaneously with the other. Further, in a perfectly viscous material, there is a phase difference between stress and strain, where strain lags behind stress by about a 90 degree phase lag. A perfectly elastic material experiences the stress and strain simultaneously such that there is not phase lag between stress and strain.

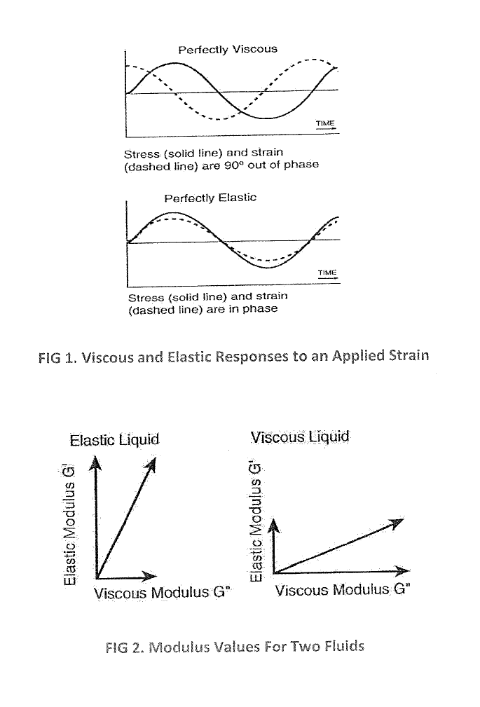

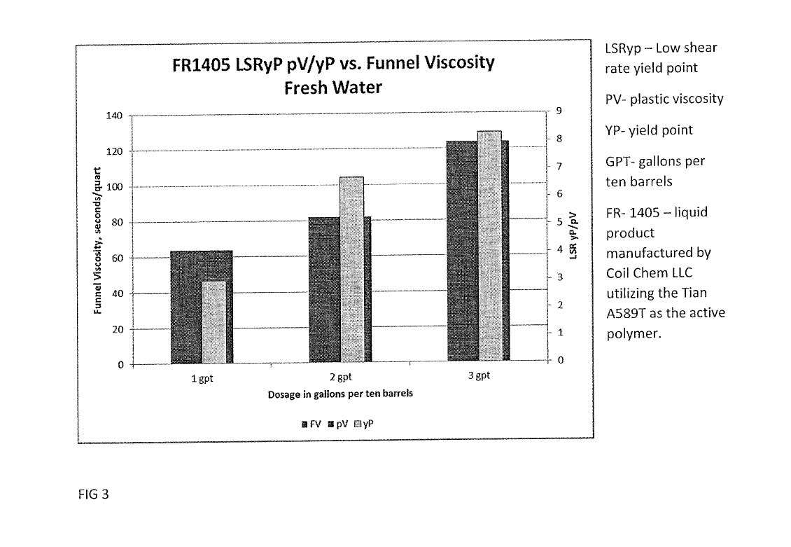

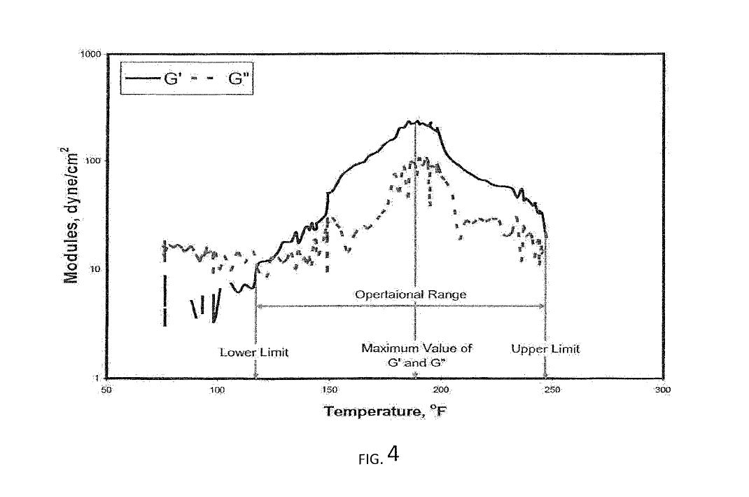

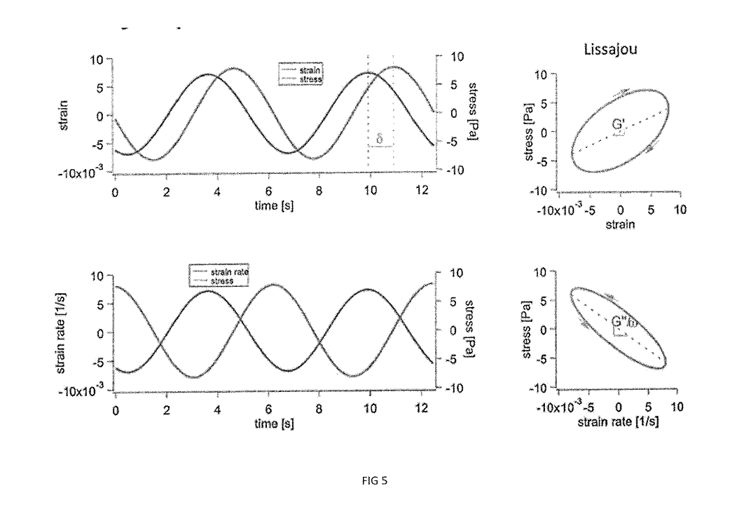

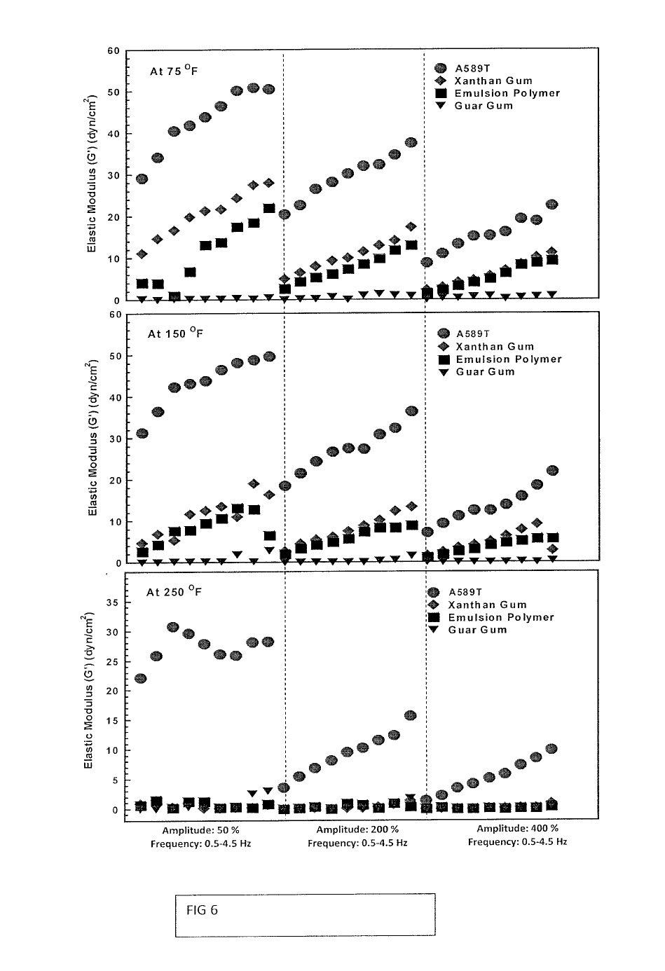

A rheometric measurement may include applying an oscillatory force to a material, such as a fluid containing a polymer, at a constant frequency, for example 1 Hz, and measuring the resulting displacement. The oscillatory force of stress may then be plotted with the displacement strain against time. FIG. 1 illustrates a typical stress response of a perfectly viscous and perfectly elastic fluid to an oscillating force (strain) on the fluid. A viscoelastic fluid may exhibit behavior somewhere in between that of a purely viscous and purely elastic material as there would be at least some phase lag θ, where 0°<θ<90°. FIG. 2 illustrates how the typical elastic and viscous modulus may plotted for an elastic and viscous fluid.

In an embodiment, a viscoelastic fluid may comprise properties of both a viscous fluid and an elastic solid. In some embodiments, the polymer included in the viscoelastic fluid may be tuned to have particular viscous and elastic properties in solution. The polymer of this disclosure may be tuned to, for example, have a high degree of elasticity. A particular polymer may comprise properties such as high shear thinning combined with a high viscoelasticity. Such a polymer in solution may not suspend particles while static as the viscosity may be too low but may lower turbulence while flowing to reduce pressure loss and horsepower requirements during flow. Furthermore, if a polymer comprises a sufficient viscoelastic component, under dynamic flow conditions in a horizontal flow conduit, the polymer may be capable of suspending particulates. In such a flow, the particulates may move as an entire mass with the fluid. In effect, the particulates may be dragged along by the polymer in the fluid. The ability to suspend particulates may be governed by the elastic memory of the polymer in the fluid. As the fluid containing the polymer flows through a conduit, eddy currents and subsequent turbulence may impart energy into the polymer. The polymer may be yielded during flow and the energy buildup subsequently released. This rebound effect may be about equal and about opposite of the energy imparted in the fluid and therefore may effectively suspend particulates within the fluid. Therefore, the polymer allows particulates (i.e. sand) to be transported at low velocities using the elastic modulus.

As the fluid and polymer flow, the rebound and release of energy stored in the polymer may cause surface disturbances between the flowing fluid and the conduit though which it is flowing. The disturbances may disrupt the interface between the moving body of fluid and the particulates that settle to the lower portion of the fluid bed. Disrupting the interface may allow the particulates to remain fluidized rather than settling out of the flow stream. A viscoelastic fluid with sufficiently high viscoelasticity may under dynamic condition flow horizontally though a conduit such that the entire mass of suspended particulates moves in conjunction with the fluid. Furthermore, disrupting the interface may reduce pipe friction thereby decreasing horsepower requirements for a pump.

Disturbing the interface between the flowing fluid and particulates suspended therein may keep the particulates fluidized and flowing with the bulk fluid rather than settling out. The loading or mass of particulate per unit volume of fluid may be increased to a larger amount than may be possible using conventional polymers that do not have the elastic rebound property as previously described.

Once the particulates settle out of solution, they may collect and plug the fracture or other flow paths. If too much plugging occurs, further particulate transport may not be possible leading to a condition known as a screenout. Screenout may cause a sudden and significant restriction to flow causing a potentially dangerous rise in pump pressure. The screenout may occur in any area with a restricted flow area such as perforations in the casing or within fractures. The polymer may reduce the amount of particle settling especially at relatively lower velocities thereby potentially reducing the conditions that enable a screenout to occur.

In an embodiment, a fluid having extremely high shear thinning characteristics may also poses extreme viscoelastic characteristics. Such a fluid may under static conditions not suspend sand or particles. However, the fluid may be used to allow a lower pump pressure thereby improving the horsepower transfer during pumping operations. Additionally, if the viscoelasticity is high enough, under dynamic conditions in a horizontal flow conduit, a mass of particles disposed in the fluid may form a mass that may move in conjunction with the fluid. This is thought to occur when the elastic memory of the material is yielded, and the energy is released. This rebound is equal to and opposite of the energy that is imparted into the fluid media and is best represented in the form of G prime and G double prime.

A relationship between stress and strain for an elastic solid may be defined by the following equations:

σ=Gγ (1)

γ=γ₀ sin(ωt) (2)

A relationship between stress and strain rate of a viscous fluid may be defined by the following equations:

σ=η\dot{γ} (3)

\dot{γ}=γ₀ω cos(ωt) (4)

A viscoelastic fluid contains properties of both a viscous liquid and an elastic solid so the previous equations may be combined to derive an equation describing viscoelastic fluids.

$$\begin{matrix}σ ( ω , t ) = {G}^{′} {γ}_{0} sin ( ω t ) + {G}^{″} {γ}_{0} sin ( ω t ) & ( 5 ) \\ η = \frac{{G}^{″}}{ω} & ( 6 )\end{matrix}$$

The term G′ represents the elastic modulus, and the term G″ represents the viscous modulus. Typically, the units of G′ and G″ are in dyn/cm² or Pascals. Equation 5 may be used to calculate G′ and G″. Without being limited by theory, it is to be understood that G′ and G″ are not temperature dependent and may be varied by temperature.

Furthermore the term σ represents sinusoidal stress with units of dyn/cm², the term G represents elastic modulus with units of dyn/cm², the term γ represents sinusoidal strain, the term γ₀ represents strain amplitude, the term ω represents angular frequency with units of 1/s, the term t represents time with units of seconds, the term η represents dynamic viscosity with units of Pa·s, and the term \dot{γ} represents strain rate with units of 1/s.

A polymer included in the fracturing fluids may have a sufficiently large G′ such that particles may be suspended by flow. A suitable polymer is available from Tianfloc Canada Inc. under the name A589T. In particular, G′ (elastic modulus) may have a value within the range of about 0.2 dyn/cm² to about 50 dyn/cm²; alternatively, about 0.2 dyn/cm² to about 10 dyn/cm², about 10 dyn/cm² to about 20 dyn/cm², about 20 dyn/cm² to about 30 dyn/cm², about 30 dyn/cm² to about 40 dyn/cm², about 40 dyn/cm² to about 50 dyn/cm², alternatively about 0.1 dyn/cm² to less than about 60 dyn/cm². Furthermore, G″ (viscous modulus) may have a value of about 0.2 dyn/cm² to about 13 dyn/cm²; alternatively, about 0.2 dyn/cm² to about 1 dyn/cm², about 0.2 dyn/cm² to about 2.0 dyn/cm², about 2.0 dyn/cm² to about 5.0 dyn/cm², about 5.0 dyn/cm² to about 8.0 dyn/cm², about 8.0 dyn/cm² to about 10.0 dyn/cm², about 1 dyn/cm² to about 3 dyn/cm², about 3 dyn/cm² to about 6 dyn/cm², about 6 dyn/cm² to about 10 dyn/cm², or about 10 dyn/cm² to about 13 dyn/cm². In some examples, G′ may be greater than about 30 dyn/cm².

A hydraulic fracturing fluid may be designed using the previously discussed polymer. There may be several design considerations taken into account during the design process of a fracturing fluid. Some parameters may include the ability of the fluid to transport a proppant, compatibilities of the fluid to the formation, pressure loss due to friction, and cost, among many others. As previously discussed, the fracturing fluid comprising the polymer may have the ability to transport proppant without the use of a viscosifying agent or turbulence. A method for designing the hydraulic fracturing fluid may comprise calculating the desired polymer amount such that the elastic component G′ is sufficiently large to be able to transport a desired amount of proppant.

A method of designing a hydraulic fracturing fluid may comprise calculating an elastic modulus of a polymer and calculating a critical velocity of a hydraulic fracturing fluid comprising the polymer and proppant based on the elastic modulus of the polymer. The calculations may be used during a hydraulic fracturing operation to ensure the fluid velocity in a tubular or fracture is above the critical velocity to prevent proppant settling. The method may further comprise calculating a maximum loading of proppant based on a concentration of the polymer and the elastic modulus. The concentration of polymer may be selected based on a desired loading of proppant. The method may further comprise a step of calculating a hydration time for the polymer. As previously discussed, the hydration time may be important as the polymer should hydrate as quickly as possible to increase the effectiveness of the polymer.

A method of hydraulic fracturing may comprise calculating an elastic modulus of a polymer, calculating a critical velocity of a hydraulic fracturing fluid comprising the polymer and a proppant based on the elastic modulus of the polymer, preparing the hydraulic fracturing fluid comprising the polymer and the proppant, and injecting the hydraulic fracturing fluid through a tubular and into a subterranean formation at a volumetric rate such that a fluid velocity in the tubular is at or above the critical velocity.

The polymer of the present disclosure may enable particulates to be transported with the fluid at much lower velocities than a standard friction reducer. Without limitation, such a velocity may enable the sand to progress much further into the fractured cavity thereby increasing the amount of area that is connected to the conduit. This increased area may result in higher initial production rates and better overall cumulative production.

In some embodiments, the use of a polymer that may move larger sand into the cavity along with higher volumes of sand may create additional surface area that is generated by the greater particle transportation. Sand loading may then be increased for longer periods of time to compensate for the increase in surface area, which may facilitate the well to be far more productive. In some embodiments, sand loading may be about 0.25 lb/gal to about 1.0 lb/gal (i.e., A589T dosage was 1.0 ppt (lbs per 1,000 gallons) to 2.0 ppt.

Without being limited by theory, the polymer may absorb and release energy in a fluid. This storing and releasing of energy within the fluid may set up surface disturbances that disrupt the interface between the moving body of fluid and the sand bed on the lower portion of the fluid bed. This building and releasing of energy within the fluid may fluidize heavy concentrations of sand and other particles allowing a dense media to be transported at velocities below the critical transport velocities as compared to conventional fluids that do not comprise the polymer. The critical transport velocities are based on maintaining mechanical agitation of the sand through the use of turbulent flow. This turbulence may then be used to help transport or carry the particles with the fluid as the fluid travels horizontally. A challenge with the use of turbulence for fluidization of particulates is that once the rate or velocity drops below turbulent levels the particulates may settle and may separate from the fluid. This settlement and separation may occur as the surface area of the formation or fractured area increases. In some embodiments, the critical velocities may be as low as 1,700 feet per min.

### Examples

Samples were prepared and tested for G′ and G″. Viscoelastic measurements were taken at various amplitude of about 50% to about 400%, frequency of about 0.5 Hz to about 4.5 Hz, and temperature off about 75° F. to about 250° F.

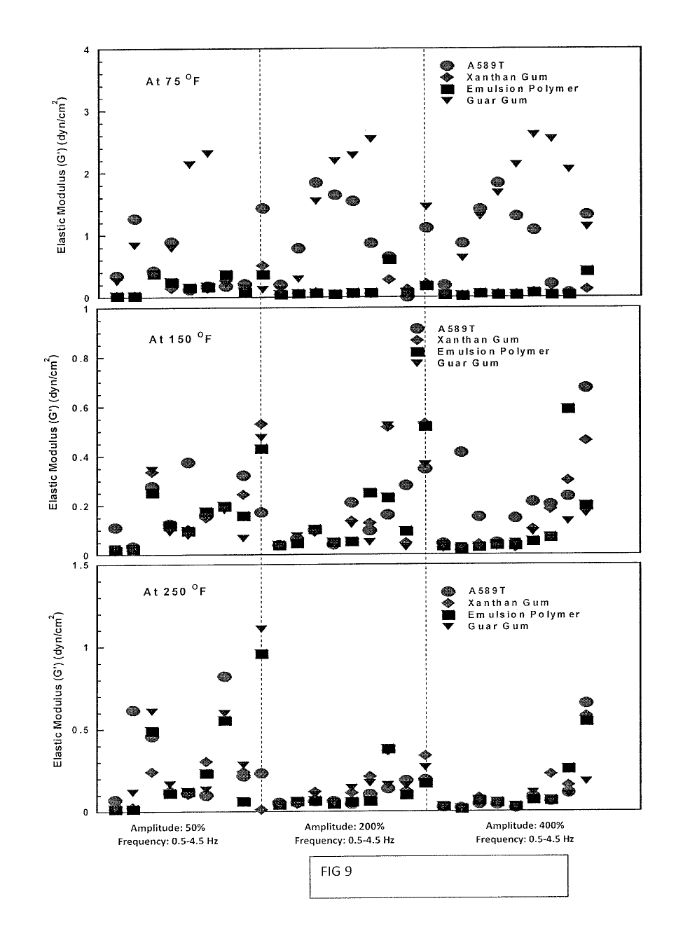

FIG. 6 illustrates elastic modulus (G′) of A589T, xanthan gum, an emulsion polymer, and guar gum in DI (Deionized) water. The samples were tested at various amplitudes, frequencies, and temperatures. A589T indicated relatively high values of elasticity comparatively to the other chemicals tested. Additionally, A589T had higher heat resistance as compared to the other tested chemicals. FIG. 7 illustrates the elastic modulus (G″) of the same samples tested at the same conditions.

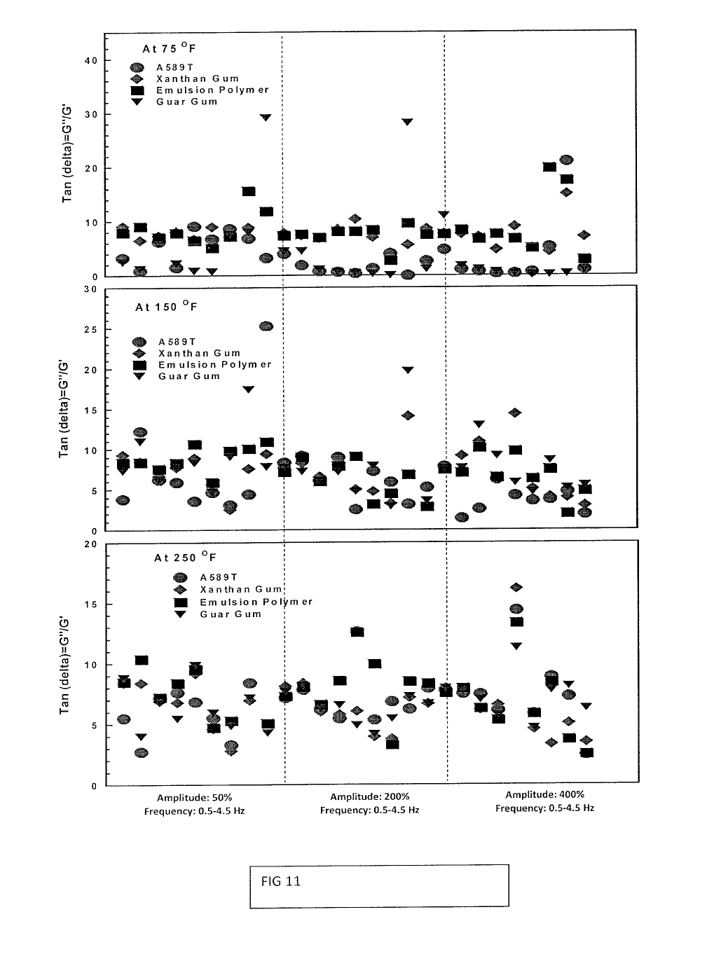

FIG. 8 illustrates the Tan(δ) comparison of the samples from FIG. 6 and FIG. 7. When the elastic modulus value is larger than the viscous modulus value, Tan(δ) becomes below 1. The lower the value of Tan(δ) is, the more elastic property the polymer has. As illustrated in FIG. 8, A589T has the highest elastic properties among the tested chemicals. The Tan(δ) may be calculated by the following equation.

$$Tan ( δ ) = \frac{Viscous Modulus ( {G}^{″} )}{Elastic Modulus ( {G}^{′} )}$$

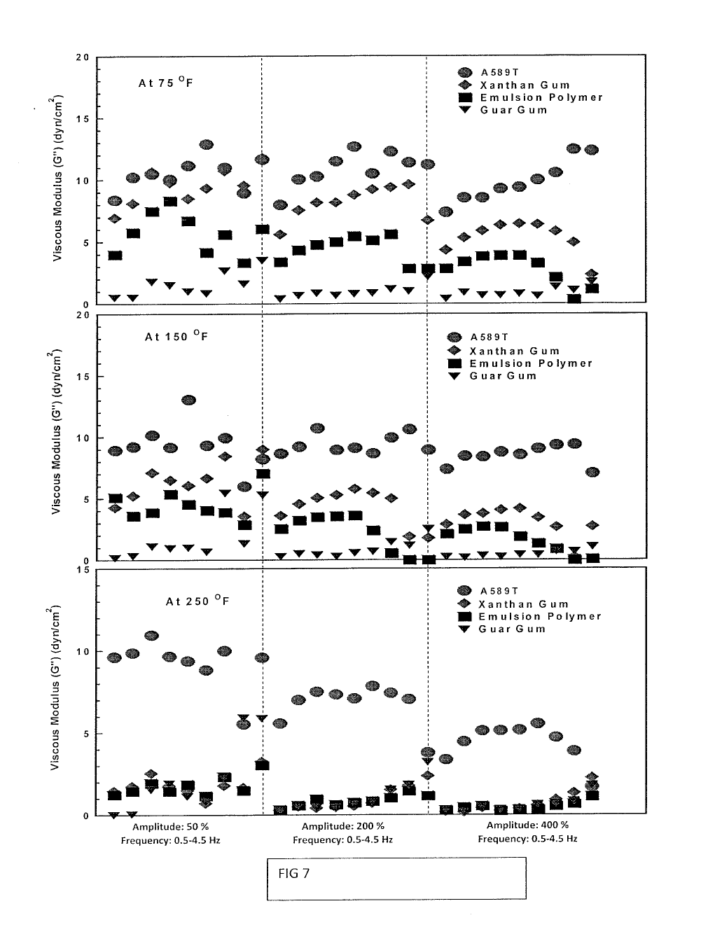

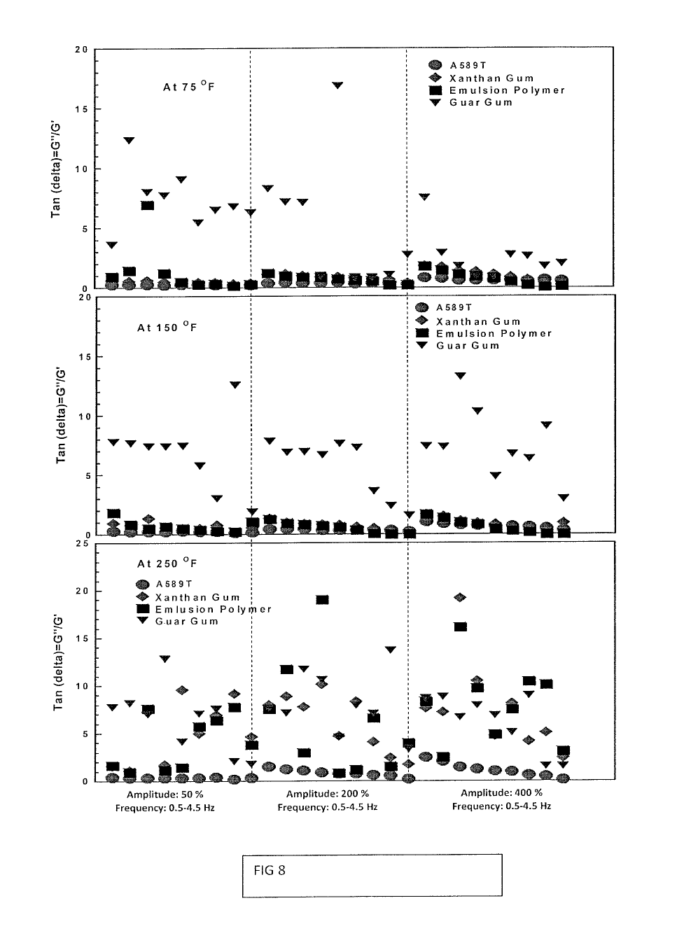

FIGS. 9, 10, and 11 illustrate the elastic modulus and viscous modulus of the same chemicals tested in FIGS. 5-8 but wherein the deionized water is replaced by 10 lb/gal brine solution. The results show that A589T has the highest elastic and viscous modulus of each of the chemicals tested.

Although the present disclosure has been described with respect to specific details, it is not intended that such details should be regarded as limitations on the scope of the invention, except to the extent that they are included in the accompanying claims. Modifications to the invention may be made as might occur to one skilled in the field of the invention within the scope of the appended claims. All embodiments contemplated hereunder that achieve the objects of the invention have not been shown in complete detail. Other embodiments may be developed without departing from the spirit of the invention or from the scope of the appended claims.

What is claimed is:

1. A method of designing a hydraulic fracturing fluid, the method comprising:
calculating an elastic modulus of a polymer; and
calculating a critical velocity of a hydraulic fracturing fluid comprising the polymer and proppant based on the elastic modulus of the polymer.

2. The method of claim 1, wherein the polymer comprises an elastic modulus of about 0.2 dyn/cm² to about 50 dyn/cm².

3. The method of claim 1, wherein the polymer comprises a viscous modulus of about 0.2 dyn/cm² to about 13 dyn/cm².

4. The method of claim 1, further comprising calculating a maximum loading of proppant based on a concentration of the polymer and the elastic modulus.

5. The method of claim 1, further comprising a step of calculating a hydration time for the polymer.

6. A method of hydraulic fracturing comprising:
calculating an elastic modulus of a polymer;
calculating a critical velocity of a hydraulic fracturing fluid comprising the polymer and a proppant based on an elastic modulus of the polymer;
preparing the hydraulic fracturing fluid comprising the polymer and the proppant; and
injecting the hydraulic fracturing fluid through a tubular and into a subterranean formation at a volumetric rate such that a fluid velocity in the tubular is at or above a critical velocity.

7. The method of claim 6, wherein the polymer comprises acrylamide.

8. The method of claim 6, wherein the polymer comprises acrylic acid, sodium acrylate, vinyl alcohol, vinyl acetate, N-vinylpyrolidone, 2-hydroxymethy methacrylate, or combinations thereof.

9. The method of claim 6, wherein the hydraulic fracturing fluid further comprises 2-acrylamido-2-methylpropane sulfonic acid.

10. The method of claim 6, wherein the polymer is provided as a granular solid comprising an average particle size of about 60 mesh to about 100 mesh.

11. The method of claim 6, further comprising calculating a maximum loading of proppant based on a concentration of the polymer and the elastic modulus.

12. The method of claim 6, further comprising a step of calculating a hydration time for the polymer.

13. The method of claim 12, wherein during the step of preparing the hydraulic fracturing fluid, the polymer is allowed to hydrate based on the calculated hydration time.

14. A hydraulic fracturing fluid comprising:
an acrylamide-based polymer having an elastic modulus greater than about 30 dyn/cm²;
water; and
a proppant.

15. The hydraulic fracturing fluid of claim 14, wherein the acrylamide based polymer further comprises acrylic acid, sodium acrylate, vinyl alcohol, vinyl acetate, N-vinylpyrolidone, 2-hydroxymethy methacrylate, or combinations thereof.

16. The hydraulic fracturing fluid of claim 14, wherein the hydraulic fracturing fluid comprises between about 1.0 wt. % and about 50 wt. % polymer.

17. The hydraulic fracturing fluid of claim 14, further comprising 2-acrylamido-2-methylpropane sulfonic acid.

18. The hydraulic fracturing fluid of claim 14, further comprising a cross linker, wherein the crosslinker comprises N,N′-methylenebis(acrylamide), boric acid, ethylene glycol diacrylate and polyethylene glycol diacrylate, or combinations thereof.

19. The hydraulic fracturing fluid of claim 14, further comprising a reaction initiator, wherein the reaction initiator comprises ammonium persulfate, benzoyl peroxide, potassium persulfate, or combinations thereof.

20. The hydraulic fracturing fluid of claim 14, further comprising a breaker, wherein the breaker comprises ammonium persulfate, sodium persulfate, sodium hypochlorite, a perborate, a peroxide, an enzyme, or combinations thereof.
